(19) United States
(12) Patent Application Publication
(10) Pub. No.: US 2017/0101375 A1
(43) Pub. Date: Apr. 13, 2017

(54) PROCESS FOR PRODUCING QUATERNARY AMMONIUM CATIONS AND IONIC LIQUIDS

(71) Applicant: CoorsTek Fluorochemicals, Inc., Arvada, CO (US)

(72) Inventors: Joseph C. Poshusta, Broomfield, CO (US); Andrew R. Riscoe, Denver, CO (US)

(73) Assignee: COORSTEK FLUOROCHEMICALS, INC., Arvada, CO (US)

(21) Appl. No.: 14/879,010

(22) Filed: Oct. 8, 2015

(51) Int. Cl. C07D 207/267

(52) U.S. Cl. CPC C07D 207/267

(57) ABSTRACT

The present invention provides a process and a method for producing a quaternary ammonium cation in an aqueous solution while reducing or preventing formation of a protonated ammonium cation. In particular, the quaternary ammonium cation is produced by adding a base to an aqueous reaction mixture comprising a tertiary amine compound and an alkylating agent.

### STATEMENT REGARDING FEDERALLY FUNDED RESEARCH

This invention was made with government support under grant number IIP-1152040 awarded by the National Science. The government has certain rights in the invention.

### FIELD OF THE INVENTION

The present invention relates to a process for producing quaternary ammonium cation in an aqueous solution. In particular, the quaternary ammonium cations that are produced by the method of the invention are useful in producing ionic liquids.

### BACKGROUND OF THE INVENTION

Quaternary amine compounds, i.e., compounds comprising a quaternary amine cation, are useful in a wide variety of applications including, but not limited to, production of ionic liquids, surfactants, catalysts, etc. Accordingly, various methods have been developed for synthesizing quaternary amine compounds. One of the methods for synthesizing quaternary amine compounds, such as pyrrolidinium bromide salts, utilizes water as a solvent. Use of water as a solvent has been shown to have numerous advantages. In general, synthesis of quaternary amine compounds involves an alkylation of a tertiary amine with an alkylating agent, such as an alkylhalide.

While use of water as a solvent simplifies the process, a side reaction involving hydrolysis of the alkylhalide (i.e., haloalkane) can lead to an undesired byproduct, e.g., a protonated ammonium compound. In some cases, separation of the protonated amine compound from the quaternary amine compound can be difficult. In fact, the quaternary amine compound is often used as a mixture containing the protonated tertiary amine compound contaminant. While the presence of a protonated amine compound in the quaternary amine compound is not detrimental in many instances, there are instances where the presence of a protonated amine compound in the quaternary amine compound can have a significantly negative impact on its use.

Therefore, there is a need for a method for producing a quaternary amine compound in an aqueous reaction solution without producing any significant amount of protonated amine compound.

### SUMMARY OF THE INVENTION

Some aspects of the invention provide a method for reducing the formation of a protonated tertiary amine cation during a process for producing a quaternary ammonium cation in an aqueous solution. The method generally includes adding a base to an aqueous reaction mixture comprising a tertiary amine and an alkylating agent. The base that is used in the method of the invention is selected such that the pKa of a counter acid of said base is higher than the pKa of said protonated tertiary amine compound. This allows the equilibrium to favor formation of the deprotonated tertiary amine compound by the base. In some embodiments, the pKa of said counter acid of said base is higher than the pKa of said protonated amine compound by at least about 1 often by at least about 2, and most often by at least about 3. The term “about” refers to ±20%, typically ±10%, and often ±5% of the numeric value.

Compared to a similar reaction condition in the absence of said base, the method of the invention typically results in at least about 80% or less, typically at least about 85% or less, often at least about 90% less, and most often at least about 95% less amount of said protonated tertiary amine compound produced and/or within the isolated quaternary ammonium cation.

In one particular embodiment, said base comprises an alkaline metal hydroxide, an alkaline earth metal hydroxide, a transition metal hydroxide, an alkaline metal oxide, an alkaline earth metal oxide, a transition metal oxide or a combination thereof. Exemplary bases that are useful in the method of the present invention include, but are not limited to, sodium hydroxide, potassium hydroxide, calcium hydroxide, lithium hydroxide, cesium hydroxide, magnesium hydroxide, strontium hydroxide, barium hydroxide, lithium oxide, sodium oxide, potassium oxide, cesium oxide, magnesium oxide, calcium oxide, strontium oxide, barium oxide or a combination thereof.

Other aspects of the invention provide a process for producing an ionic liquid comprising a quaternary amine cation. Such a process generally includes:
(a) adding a base to an aqueous reaction solution comprising a tertiary amine compound and an alkylating agent to produce a cationic portion of said ionic liquid, wherein said cationic portion of said ionic liquid is a quaternary amine compound;
(b) removing at least a portion of said tertiary amine compound from the aqueous reaction solution to produce a separated quaternary amine compound; and
(c) admixing said separated quaternary amine compound with the anionic portion of said ionic liquid to produce said ionic liquid.

In this manner, the amount of ionic liquid compound produced from protonated tertiary amine compound is significantly reduced compared to a process that does not include adding a base to remove the tertiary amine compound. The resulting ionic liquid has at least about 80% or less, typically at least about 85% or less, often at least about 90% less, and most often at least about 95% less amount of ionic liquid compound containing a protonated tertiary amine compound as the cationic moiety. Alternatively, said ionic liquid having a quaternary amine cation comprises about 500 ppm or less, typically about 200 ppm or less, often about 100 ppm or less, more often about 50 ppm or less and most often about 10 ppm or less of the protonated tertiary amine ionic liquid.

In one embodiment, the pKa of a counter acid of said base is higher than the pKa of said protonated tertiary amine compound. Yet in another embodiment, said base comprises an alkaline metal hydroxide, an alkaline earth metal hydroxide, a transition metal hydroxide, an alkaline metal oxide, an alkaline earth metal oxide, a transition metal oxide or a combination thereof. Exemplary bases that are useful in the process of the invention include, but are not limited to, sodium hydroxide, potassium hydroxide, calcium hydroxide, lithium hydroxide, cesium hydroxide, magnesium hydroxide, strontium hydroxide, barium hydroxide, lithium oxide, sodium oxide, potassium oxide, cesium oxide, magnesium oxide, calcium oxide, strontium oxide, barium oxide or a combination thereof.

Still other aspects of the invention provide a process for producing a quaternary amine compound comprising:
reacting a tertiary amine compound with an alkylating agent in an aqueous reaction mixture in the presence of a base under conditions sufficient to produce a quaternary amine compound, wherein the pKa of a counter acid of said base is higher than the pKa of said protonated tertiary amine compound; and
removing at least a portion of said tertiary amine compound is removed from the aqueous reaction solution.

In some embodiments, the process further comprises the step of metathesizing said quaternary amine compound to produce an ionic liquid and removing at least a portion of said tertiary amine from the ionic liquid. Typically substantially all (e.g., at least about 90%, typically at least about 95%, and most often at least about 99%) of said tertiary amine is removed from the ionic liquid.

Typically, the process of the invention results in the quaternary amine compound in which the amount of a protonated tertiary amine compound produced in the aqueous reaction mixture is at least about 80% or less, typically at least about 85% or less, often at least 90% or less, and most often at least about 95% or less than a similar reaction condition in the absence of said base.

### DETAILED DESCRIPTION OF THE INVENTION

Synthesis of quaternary ammonium cations, such as pyrrolidinium bromide salts, in an aqueous solution has been shown to have numerous advantages. In general, these reactions involve reaction between a tertiary amine compound I and an alkylating agent II, such as an alkylhalide, to produce a quaternized ammonium cationic compound III (i.e., “quaternary ammonium cation”). The overall reaction is illustrated by the following reaction equation:

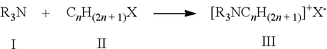

While use of water as a solvent simplifies the approach, a side reaction involving hydrolysis of the alkylating agent II, e.g., haloalkane, can lead to an undesired byproduct of a protonated amine cation. Without being bound by any theory,

It is believed that the reaction involves hydrolysis of the alkylating agent, e.g., alkylhalide II, to form the corresponding alcohol IV and the acid halide V via the following reaction:

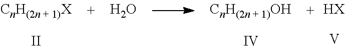

The acid halide V that is produced in the above reaction can neutralize the basic tertiary amine compound I to form the protonated tertiary amine compound (i.e., protonated tertiary ammonium halide salt) VI as illustrated in the following reaction equation:

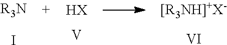

For example, bromopropane hydrolyzes slowly in water to produce HBr and propanol. The HBr that is generated in the reaction can react with N-methyl pyrrolidine (i.e., mPyr) to form hydrogen methyl pyrrolidinium bromide (“Pyr1H Br”), a protonated amine bromide salt.

The reaction to form a quaternary amine halide salt III is typically one of the first steps in preparing an ionic liquid, where the quaternary amine cation of compound III is used as the cationic portion of the ionic liquid. The desired anion is manufactured in a separate process and is typically provided as an acid or alkali metal salt of the desired anion. If the ionic liquid is hydrophobic, the desired cation and anion can be easily paired by metathesis in water. The undesired cation and anion are highly water soluble and are readily removed in the aqueous phase. The reaction scheme below illustrates a general reaction for producing an ionic liquid by metathesis in water:

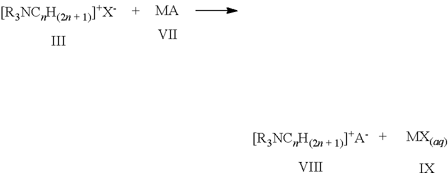

where M is a proton or mono-valent metal cation. If the resulting salt MX (compound IX) is highly water soluble, then an ionic liquid VIII can be isolated at more than 95% purity. Any protonated amine compounds (e.g., compound VI) that maybe present will also participate in the metathesis as illustrated by the following reaction scheme:

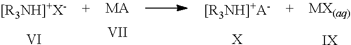

The protic cation metathesis product X may also be hydrophobic and therefore difficult to remove from the desired ionic liquid VIII by extraction with water. For example, in the metathesis of the N-methyl-N-propyl pyrrolidinium bromide (“Pyr13 Br”) with a bis(fluorosulfonyl)imide (“FSI”) salt, the presence of a small amount of N-methyl pyrrolidinium bromide (“Pyr1H Br”)—which may be present in the starting Pyr13 Br—leads to formation of N-methyl pyrrolidinium bis(fluorosulfonyl)imide (“Pyr1H FSI”), which is also an ionic liquid. The presence of this byproduct (Pyr1H FSI) is low in concentration, but its removal is very difficult. For example, attempts to remove Pyr1H FSI from Pyr13 FSI via extraction with water have shown to be inefficient. Attempts to remove Pyr1H FSI by adsorption on carbon have also been shown to be ineffective.

The presence of protonated amine compounds can be detected and quantified by electrochemical methods or by cation chromatography. In addition, on cyclic voltammetry, the integrated area of the current density versus voltage curve for the reduction can be correlated to the concentration of protonated amine compound. The protonated amine compound can be reduced on a platinum or glassy carbon electrode during cyclic voltammetry. This electrochemical reducibility of protonated amine compound renders its presence in electrolytes undesirable when used in batteries and capacitors.

Some aspects of the invention provide a method for reducing or eliminating formation of a protonated tertiary amine compound while synthesizing a quaternary amine compound using an aqueous reaction solution. As used herein, the term “protonated amine compound” refers to an amine compound in which the nitrogen atom of the amine compound is protonated. Typically, the protonated amine compound is a tertiary amine compound that is protonated (i.e., “protonated tertiary amine compound”). However, it should be appreciated that the scope of the invention is not limited to tertiary amine compounds as methods of the invention can also be used to reduce or prevent formation/production of protonated monoalkyl and dialkyl amine compounds during an alkylation reaction in an aqueous solution. The term “amine compound” refers to a compound having a basic nitrogen atom with a lone pair. Amine compounds are derivatives of ammonia, wherein one or more hydrogen atoms have been replaced by a hydrocarbon substituent such as an alkyl or aryl group or any other hydrocarbon moieties known to one skilled in the art. It should be appreciated that two or more hydrocarbon moieties that are attached to the nitrogen atom of the amine compound can form a ring system. “Hydrocarbon” can include one or more unsaturations, e.g., carbon-carbon double bond or triple bond. Hydrocarbon can be a straight chain, branched chain, mono- or bi-cyclic ring system, or a combination thereof. In addition, hydrocarbon can also include other functional groups, such as ethers, hydroxides (protected and/or unprotected), esters, carboxylic acids, sulfates, etc. as long as the functional group does not substantially interfere with alkylation reaction between an alkylating agent and the amine functional group. The nitrogen atom of the amine compound can be a part of a heterocyclic ring system or a heteroaryl ring system. Exemplary heterocyclic ring systems having one or more nitrogen atoms include, but are not limited to, piperidinyl, piperazinyl, morpholinyl and thiomorpholinyl, etc. Exemplary heteroaryl ring systems having one of more nitrogen atoms include, but are not limited to, pyridyl, triazolyl, imidazolyl, isoxazolyl, pyrrolyl, pyrazolyl, pyrimidinyl, benzotriazolyl, indolyl, isoindolyl, benzoxazolyl, benzimidazolyl, etc.

In some embodiments of the invention, the protonated amine compound is an ionic liquid. The term “ionic liquid” refers to a salt in which the ions are poorly coordinated, which results in these compounds being liquid below, for example, 100° C., or even at room temperature (i.e., room temperature ionic liquids or RTIL's) or below. Ambient temperature (i.e., room temperature or about 20° C.) ionic liquids are often used as electrolytes. Ionic liquids are non-volatile, non-flammable, has a wide electrochemical stability window and high ionic conductivity. The terms “quaternary amine compound” and “quaternary amine cation compound” and “quaternary amine cation” are used interchangeably herein and refer to a nitrogen atom containing compound in which the nitrogen atom is attached to four hydrocarbon moieties or carbon atoms. Thus, as used herein, the term quaternary amine compound explicitly excludes protonated amine compounds, such as protonated tertiary amine compounds and the like. Typically, the quaternary amine compound is derived from alkylation of an unprotonated amine compound.

Some aspects of the invention provide a method for producing a quaternary ammonium cation using an aqueous solvent. In particular, the quaternary ammonium cation produced by the method of the invention has a significantly less amount of protonated tertiary amine compound impurities compared to conventional methods. In one particular aspect of the invention, the method includes adding a base to a reaction mixture comprising a tertiary amine and an alkylating agent. It should be appreciated that the order of adding the base, the tertiary amine compound, and the alkylating agent does not matter. In general, the base is added to the mixture of the tertiary amine compound and the alkylating agent. However, the scope of the invention includes any order of adding each compound.

Typically, the base has significantly stronger basicity than the counter acid of the tertiary amine compound. In this manner, addition of the base to the aqueous reaction mixture reduces or in sufficient amount eliminates the production of protic or protonated tertiary amine compound. Often, while the basicity of the base may be stronger than the basicity of the counter acid of the tertiary amine compound, the nucleophilicity of the base is weaker than the nucleophilicity of the tertiary amine. Thus, the nucleophilic substitution reaction between the tertiary amine compound and the alkylating agent is significantly faster (i.e., higher kinetic rate) than the nucleophilic substitution reaction between the base and the alkylating agent. Since hydrolysis of bromoalkanes is catalyzed in either base or acid, one might expect this hydrolysis to be accelerated in the presence of strong base in the reaction mixture. However, it was found by the present inventors that the presence of a strong base did not adversely affect the kinetic rate of nucleophilic substitution reaction between the tertiary amine and the alkylating agent, and thus the production of the quaternary ammonium cation.

Without being bound by any theory, it is believed that addition of a strong base in high enough concentration to the water based alkylation of a tertiary amine raises the pH of the reaction mixture sufficiently to prevent or significantly reduce the amount of a protonated tertiary ammonium cation formation. The production of the protonated ammonium cation is reversible and the equilibrium of the reaction is pH dependent. High pH favors formation of a neutral tertiary amine compound of the following reaction equation:

[R₃NH]⁺ [symbol] R₃N+H⁺

For many tertiary amines, the equilibrium constant (Kₐ) of this reaction is known. For example, N-methyl pyrrolidine (“mPyr”) has a pKb of 3.45 (pKa=10.6). The equilibrium relationship between the protonated form and the deprotonated form is given by the following equation:

Kₐ═([H⁺]×[R₃N]/[R₃NH⁺]

It should be noted that in the above equation, brackets represent concentration of the species in molarity. Rearranging for the concentration of protons yields the following equation:

10^{-pH}=[H⁺]=Ka×([R₃NH⁺]/[R₃N])

It is desired to reduce the ratio of protonated amine compound to amine base, i.e., the unprotonated amine compound. Thus, the pH of the system should be larger than pKₐ of the protonated amine compound in order to have a ratio of protonated amine compound to tertiary amine base that is less than 1, typically less than 0.5, often less than 0.1, and most often less than 0.01. In a system where the pH is substantially less than pKₐ of the protonated amine compound, the protonated amine compound will be stable and remain protonated. If the pH conditions that favor the forward deprotonation of the protonated amine compound are maintained after the alkylation reaction to produce the quaternary amine compound, then the protonated amine compound will be deprotonated and converted back to the tertiary amine compound. For example, at a pH of 11.6 the concentration of protonated amine compound is about 1/10 of the mPyr concentration and at pH of 12.6, the ratio decreases to 1/100.

The pH of the reaction solution can be increased by adding a base substantially stronger than the tertiary amine compound. For example, hydroxides, such as alkali metal hydroxides and alkaline earth metal hydroxides, are strong bases that can raise the pH with a small addition. When the base is added at the beginning of the reaction, a sufficiently high pH can be maintained throughout the alkylation reaction, thereby substantially reducing the amount of protic ammonium cation formed in the reaction. It is believed that some hydroxide base is consumed by hydrolysis of alkyl halide to generate water and an alkali metal salt. If the hydrolysis reaction is slow relative to the alkylation of the tertiary amine, then the effect of lost base will also be small.

Using methods of the invention, where the alkylation reaction is conducted under basic conditions, the resulting solution contains primarily a desired quaternary ammonium cation salt and water. Depending on the amount of base added, there may also be a small amount of excess alkali metal hydroxide, excess alkyl halide precursor, and an alcohol and alkali metal halide salt from hydrolysis of the alkyl halide. When used to make a hydrophobic ionic liquid in a metathesis reaction, traces of these contaminants may remain in the ionic liquid phase. Subsequent extraction with water removes the water-soluble species (e.g., excess alkali metal hydroxide and alkali metal halide salt) effectively. However, the excess alkyl halide and corresponding alcohol partition into the ionic liquid phase and are not removed effectively by extraction with water. However, short chain alkyl halides have a high vapor pressure and can be removed from the ionic liquid by simple distillation or vapor stripping methods or by adsorption on activated carbon.

Other alkylating agents that are useful include, in general, any alkyl groups with a leaving group in which the counter acid of the leaving group has a pKa of no more than 15, typically no more than 10, often no more than 5, and most often no more than 1. “Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like. Exemplary alkylating groups that are suitable for methods of the invention include, but are not limited to, alkyl groups having the following leaving groups: halide (such as chloride, bromide, or iodide), alkylsulfates, alkanesulfonyloxy, arenesulfonyloxy, etc. Some of the specific leaving group of alkylating agents that are useful in the present invention include, but are not limited to, chloride, bromide, iodide, sulfonate, mesyloxy, tosyloxy, etc. For example, dialkylsulfates are well known alkylating agents that can be used as an alkylating agent as shown in the following reaction equation:

(CₙH₍₂ₙ₊₁₎)₂SO₄+R₃N→[CₙH₍ₙ₊₁₎NR₃]⁺.⁻SO₄CₙH₍₂ₙ₊₁₎

Methods of the invention produces quaternary amine cation having less than 200 ppm, typically less than 50 ppm, and often less than 10 ppm of the protonated amine compound (i.e., protonated ammonium cation) in a similar reaction condition in the absence of the base.

Some aspects of the invention provide a quaternary amine ionic liquid that has a minute amount of protonated tertiary ammonium ionic liquid as an impurity. That is the ionic liquid comprising a quaternary ammonium cation and a suitable ionic liquid anion can be produced using the methods or the processes of the invention. The quaternary ammonium cation is produced using the methods described herein, whereas the appropriate anionic portion of the ionic liquid can be produced using any of the methods known to one skilled in the art including commonly assigned U.S. patent application Ser. Nos. 13/598,570 and 13/951,973, which are incorporated herein by reference in their entirety. Such quaternary amine ionic liquids of the invention have less than 10 ppm, typically less than 5 ppm, and often less than 1 ppm of protonated tertiary ammonium ionic liquid.

In some embodiments, the quaternary amine compound is used to produce an ionic liquid. In some instances, the ionic liquid is a room temperature ionic liquid at a standard condition. The term “standard condition” refers to 1 atmospheric pressure at 20° C.

In producing an ionic liquid comprising a quaternary ammonium cation disclosed herein, typically the quaternary ammonium compound is subjected to a metathesis reaction with a corresponding anion of the ionic liquid. The quaternary amine compound produced from alkylation is often isolated and purified before being used to produce an ionic liquid. However, it should be noted that the purification step is not necessary at this stage, as the purification step can be carried out after making the ionic liquid.

In some instances, the quaternary ammonium compound can be purified to further remove any protonated tertiary ammonium cation that may be present. Such a purification can be achieved according to the methods disclosed in a commonly assigned U.S. Provisional patent application that is filed even date with this application and is further identified by the Attorney Docket No. ION-000800PV.

For example, in preparing an ionic liquid, the base can be added to the quaternary amine compound prior to the metathesis reaction to remove the protonated amine compound. Alternatively, or in addition, the base can be added to the quaternary amine compound after the metathesis reaction to remove the protonated amine compound. Regardless of when the base is added, in some instances the quaternary amine compound and the base are admixed in an aqueous solution to remove the protonated amine compound. The addition of base to the quaternary amine compound deprotonates protonated amine compound that may be present as a contaminant or impurity in the quaternary amine compound. Deprotonation of the protonated amine compound converts the protonated amine compound to its corresponding neutral amine compound. Since the amine compound that is produced from deprotonation of the protonated amine compound is neutral, i.e., non-ionic, it will have a significantly different physical properties including a significantly higher vapor pressure, thereby allowing a much easier separation from the quaternary amine compound.

In some embodiments, the amine compound produced from deprotonation is removed (i.e., separated) from the quaternary amine compound by adsorption. For example, amine compound is readily adsorbed by carbon (e.g., activated charcoal, etc.), whereas quaternary amine compounds are not readily adsorbed by carbon due to its ionic nature. Moreover, since the amine compound has a significantly higher vapor pressure, it can be removed or separated from the quaternary amine compound as a vapor. For example, the amine compound can be distilled (e.g., under vacuum) away from the quaternary amine compound. It can also be removed from quaternary amine compound by gas stripping, e.g., the amine compound is removed from the quaternary amine compound by flowing a stream of gas (e.g., nitrogen, air, helium, etc.). Such a stream of gas can be bubbled through a solution of quaternary amine compound. If the quaternary amine compound is an ionic liquid, the stream of gas can be bubbled directly into the quaternary amine compound. Alternatively, or in addition, the stream of gas can be flowed across the surface of the quaternary amine compound or its solution.

As discussed above, an ionic liquid is a salt that is a liquid at temperature below 100° C., and often at room temperature. The ionic liquid of the invention comprises a quaternary amine cation component and an anion component. In some instances, the anion component is bis(fluorosulfonyl)imide (“FSI”).

In some embodiments, the ionic liquid comprises about 20 ppm or less, typically 10 ppm or less, and often 1 ppm or less of said protonated amine compound.

Some aspects of the invention provide an ionic liquid that comprises a quaternary ammonium cation and a corresponding ionic liquid anion (e.g., FSI), where the ionic liquid has less than 20 ppm, typically less than 10 ppm, and often less than 1 ppm of an impurity ionic liquid that comprises a protonated tertiary ammonium cation and a corresponding ionic liquid anion. Thus, while quaternary amine ionic liquids of the invention may include a trace amount of tertiary amine ionic liquid as an impurity, the amount of tertiary amine ionic liquid is significantly less than those obtained from conventional methods.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

### Example 1: Reaction of N-Methylpyrrolidine with 1-Bromopropane and Lithium Hydroxide in Water

In a 250 mL three neck glass flask 45.5 g of N-methylpyrrolidine was added to 112 g of deionized water using a dropping funnel and maintaining a temperature lower than 31.1° C. Lithium Hydroxide monohydrate (0.939 g) was then added to the mixture. 1-bromopropane (72.3 g, 11% excess by moles) was added dropwise to the mixture over the course of 40 minutes with the maximum temperature of 68° C. during the addition. The mixture was then refluxed at 70° C. for two hours. The resultant product was mainly a solution with a few drops of a more dense second phase, presumably 1-bromopropane. A portion of the solution was mixed with 10% activated charcoal for 18 hours and filtered under vacuum. Ion Chromatography analysis of the carbon treated as well as untreated solution revealed <5 ppm Pyr_{1H}⁺.

### Example 2: Reaction of N-Methylpyrrolidine with 1-Bromopropane in Water

In a 2000 mL three neck flask, 407 g of N-methylpyrrolidine was added to 998 g deionized water from a dropping funnel maintaining a temperature lower than 40° C. 1-bromopropane (591 g) was then added via a droping funnel over the course of 45 minutes with the maximum temperature reached during addition of 68° C. The mixture was then refluxed at 88° C. for 2 hours. The resultant solution was homogeneous. A portion of the solution was mixed with 10% activated charcoal for 18 hours and filtered under vacuum. Ion chromatography analysis of the untreated and treated portion both yielded 950±25 ppm Pyr_{1H}⁺.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for reducing the formation of a protonated tertiary amine cation during a process for producing a quaternary ammonium cation in an aqueous solution, said method comprising adding a base to an aqueous reaction mixture comprising a tertiary amine and an alkylating agent, wherein the pKa of a counter acid of said base is higher than the pKa of said protonated tertiary amine compound.

2. The method of claim 1, wherein the pKa of said counter acid of said base is higher than the pKa of said protonated amine compound by at least 1.

3. The method of claim 2, wherein the pKa of said counter acid of said base is higher than the pKa of said protonated amine compound by at least 2.

4. The method of claim 1, wherein the amount of said protonated tertiary amine compound produced is at least 80% less than a similar reaction condition in the absence of said base.

5. The method of claim 1, wherein said base comprises an alkaline metal hydroxide, an alkaline earth metal hydroxide, a transition metal hydroxide, an alkaline metal oxide, an alkaline earth metal oxide, a transition metal oxide or a combination thereof.

6. The method of claim 5, wherein said base comprises sodium hydroxide, potassium hydroxide, calcium hydroxide, lithium hydroxide, cesium hydroxide, magnesium hydroxide, strontium hydroxide, barium hydroxide, lithium oxide, sodium oxide, potassium oxide, cesium oxide, magnesium oxide, calcium oxide, strontium oxide, barium oxide or a combination thereof.

7. A process for producing an ionic liquid comprising a quaternary amine cation, said process comprising:
(a) adding a base to an aqueous reaction solution comprising a tertiary amine compound and an alkylating agent to produce a cationic portion of said ionic liquid, wherein said cationic portion of said ionic liquid is a quaternary amine compound;
(b) separating said tertiary amine compound from the aqueous reaction solution; and
(c) admixing said separated quaternary amine compound with the anionic portion of said ionic liquid to produce said ionic liquid,
wherein the pKa of a counter acid of said base is higher than the pKa of said protonated tertiary amine compound.

8. The process of claim 7, wherein said base comprises an alkaline metal hydroxide, an alkaline earth metal hydroxide, a transition metal hydroxide, an alkaline metal oxide, an alkaline earth metal oxide, a transition metal oxide or a combination thereof.

9. The process of claim 8, wherein said base comprises sodium hydroxide, potassium hydroxide, calcium hydroxide, lithium hydroxide, cesium hydroxide, magnesium hydroxide, strontium hydroxide, barium hydroxide, lithium oxide, sodium oxide, potassium oxide, cesium oxide, magnesium oxide, calcium oxide, strontium oxide, barium oxide or a combination thereof.

10. The process of claim 7, wherein said ionic liquid comprises about 50 ppm or less of a protonated tertiary amine ionic liquid.

11. The process of claim 10, wherein the amount of said protonated tertiary amine ionic liquid produced is at least 80% less than a similar reaction condition in the absence of said base in said step (a).

12. A process for producing a quaternary amine compound comprising:
reacting a tertiary amine compound with an alkylating agent in an aqueous reaction mixture in the presence of a base under conditions sufficient to produce a quaternary amine compound, wherein the pKa of a counter acid of said base is higher than the pKa of said protonated tertiary amine compound; and
removing at least a portion of said tertiary amine compound is removed from the aqueous reaction solution.

13. The process of claim 12 further comprising the step of metathesizing said quaternary amine compound to produce an ionic liquid and removing at least a portion of said tertiary amine from the ionic liquid.

14. The process of claim 12, wherein the amount of a protonated tertiary amine compound produced in the aqueous reaction mixture is at least 80% less than a similar reaction condition in the absence of said base.
